(19) United States
(12) Patent Application Publication
(10) Pub. No.: US 2007/0098740 A1
(43) Pub. Date: May 3, 2007

(54) Stabilisation of viral microparticles

(76) Inventor: Christopher Iain Grainger, LONDON (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/580,769

(22) PCT Filed: Nov. 29, 2004
(86) PCT No.: PCT/GB04/04996

§ 371 Date: Aug. 9, 2006

(30) Foreign Application Priority Data
Nov. 28, 2003 (GB) 0327727.4

(51) Int. Cl. A61K 39/12; A61K 31/7012

(52) U.S. Cl. 424/204.1; 514/53; 977/802

(57) ABSTRACT

A micro-particulate dry powder composition comprising a viral particle can be prepared by spray-drying a mixture of the viral particle and a stabilizing carbohydrate using an outlet temperature of no more than 60° C.

### FIELD OF INVENTION

This invention relates to virus-containing microparticles.

### BACKGROUND TO THE INVENTION

Vaccination has been a hugely successful method of reducing the incidence of disease. For example, measles vaccination has been used routinely from the 1960s onwards and since that time, global incidence has been reduced by 72%. However, many vaccines still rely on sub-cutaneous delivery, limiting their use in the third world due to the health risks associated with needles and their safe disposal. Continuing the example, measles infection still accounts for almost one million fatalities per year globally, and is still one of the major causes of infant mortality in developing countries. Research into vaccines has therefore been redirected at alternative dosing routes.

The mucosal route is a preferred method of vaccine delivery since virus filled droplets from infected individuals enter hosts via the mucosal membranes of the upper respiratory tract. It is well known that pulmonary delivery of antigens produces a mucosal immunity superior to that which is produced by parental administration. Thus, it makes theoretical sense to vaccinate through the natural route of infection, inducing mucosal immunity to effectively interrupt the transmission of the virus.

Over 10 years ago, Sabin proposed the use of an aerosol measles vaccine in mass campaigns (Sabin A B, European Journal of Epidemology, 1991; 1:1-22). Since then, dosing via the lung using a nebulised aerosol, has been found to be preferable compared to sub-cutaneous injection.

However, nebulisation has its drawbacks. The apparatus can be cumbersome, delivered doses variable, and it presents stability issues for the vaccine once reconstituted. Other studies have investigated a dry powder approach for immunisation by inhalation (Licalsi C. et al., Vaccine, 2001; 19:2629-2636). This would theoretically provide the same immunogenic advantages of delivery to the lung mucosa as nebulisation, but circumvent the issues associated with liquid aerosolisation, thus presenting further advantages such as an ease of manufacture (compared to lyophilisation needed for nebulisation) and reduced environmental contamination.

There is therefore the need for vaccines suitable for mucosal delivery that are not dependent on nebulisation or liquid aerosolisation. Current approaches to producing vaccines suitable for inhalation and mucosal delivery focus on jet milling of conventional foam or freeze-dried live virus-containing matrices. However, milling is known to be detrimental to virus stability and recovery rates and is limited in the range of particle sizes it can produce.

There is therefore the need for vaccines suitable for inhalation and mucosal delivery that provide stable virus particles with high recovery rates in the production process.

### SUMMARY OF INVENTION

The present invention provides a novel method of producing vaccines in a dry powder format. This powder is suitable for delivery to the body via multiple routes, including (but not limited to) inhalation to the alveolar and bronchiolar regions of the lung, nasal, ocular and ballistic (into, through or across the skin) delivery routes.

According to a first aspect of the invention a method for producing a micro-particle dry powder comprising a viral particle, comprises the steps of:

spray-drying a mixture of the viral particle and a stabilising carbohydrate using an outlet temperature of no more than 60° C.

The spray-drying of virus particles is surprising as it would be expected that conventional temperatures used in spray drying (˜130° C. inlet, ˜90° C. outlet) would be too hot for most viruses to tolerate. The current invention is based on the surprising realisation that the addition of a stabilising carbohydrate to a viral particle, in combination with a novel combination of spray dryer parameters, allows the temperatures used to be decreased to less than 60° C. whilst still producing virus-containing microparticles suitable for pulmonary delivery.

This method offers an alternative strategy for vaccination, which is of greater utility than the currently favoured sub-cutaneous, nebulisation and aerosolisation methods. It is a one-step method which can provide particles of any desired size and which provides stable virus particles with high recovery rates.

A second aspect of the invention comprises a virus-containing microparticle dry powder, suitable for pulmonary delivery.

A third aspect of the invention comprises the use of a virus-containing microparticle dry powder, in the manufacture of a vaccine for the prevention of a viral infection.

### DESCRIPTION OF THE DRAWINGS

The invention is described with reference to the accompanying drawings wherein:

FIG. 1 shows the effect of outlet temperature, atomisation pressure and trehalose feedstock concentration upon Schwarz strain virus recoveries post-manufacture;

FIG. 2 shows the effect of trehalose feedstock and HSA concentration upon EZ strain recoveries virus post manufacture; and

FIG. 3 shows an SEM of particles produced from a 50% trehalose solution with 1% w/v HSA.

### DETAILED DESCRIPTION OF THE INVENTION

The present invention makes use of spray drying technology to manufacture novel microparticles comprising a viral particle, particularly suited to pulmonary delivery.

The process of spray drying is well known in the art, and involves the atomisation of a solution, suspension or dispersion of the virus containing microparticles, and then directing the resulting droplets into a drying chamber. Preferably the mixture that is dried is non-gaseous.

Any suitable spray dryer may be used. A preferred embodiment uses a 2-fluid nozzle design in a sealed chamber, to prevent contamination. Feed concentrations, pump rates, atomisation pressures and nozzle types can all be selected based on the guidelines provided herein. The atomisation and spraying stage may make use of a conventional atomisation process, e.g. pressure or two fluid nozzles, or may utilise an ultrasonic atomisation process (Maa et al., Pharmaceutical Research, 1999: 16(2)) or a rotary atomiser. The present invention specifies a unique range of parameters which, when used to spray-dry a virus/stabilising carbohydrate mix, allow the outlet temperature to be reduced to below 60° C. and produce virus-containing microparticles suitable for pulmonary delivery.

Drying will usually be carried out to achieve a residual moisture content of the microparticles of less than 10% by weight, preferably less than 5% by weight and most preferably less than 3% by weight.

To give stable virus-containing microparticles with desirable characteristics, the outlet temperature of the spray dryer should be no more than 60° C. Preferably the spray dryer has an outlet temperature from 20 to 40° C. and most preferably the outlet temperature is about 30° C.

The concentration of carbohydrate used may be determined based on the amount of virus to be stabilised and the particular carbohydrate to be used. In general, the concentration will be from 2% w/v to 70% w/v, more preferably 30% w/v to 60% w/v and most preferably 40% w/v to 55% w/v. In an alternative embodiment, the most preferred concentration of the carbohydrate is from about 6% w/v to about 12% w/v.

The feed-rate of the spray dryer is selected based on the temperature to be used. In general, the feed-rate is preferably from 0.05 to 2 grams/minute, and is most preferably about 0.25 grams/minute.

The spray dryer nozzle-kit configuration may be selected based on the temperature and the feed-rate to be used. The configuration may be from 1 bar 10L/sec to 3 bar 30L/sec. In a preferred embodiment, the configuration is from about 3 bar 10L/sec to about 3 bar 30L/sec, most preferably about 3 bar 22L/sec. In a further preferred embodiment, the nozzle configuration is from about 1.5 bar 10L/sec to about 1.5 bar 30L/sec, most preferably about 1.5 bar 14L/sec.

The drying air pressure is preferably from 1.5 bar to 3 bar, most preferably about 2 bar.

The drying air flow rate is preferably from 4.8L/sec to 8L/sec, most preferably about 6L/sec.

The atomisation airflow rate is preferably from 0.01 to 0.60L/sec, most preferably about 0.23L/sec.

The spray-drying process may also make use of high efficiency cyclones, bag filters or sintered glass filters to adjust the percentage amount of different sized particles in the resulting particulate composition.

The optimal spray dryer parameters and carbohydrate composition may also be adapted for use in the related technique of spray-freeze drying.

Microparticles suitable for pulmonary delivery will usually have a mean aerodynamic particle diameter size ranging from 0.1 to 40 μM, preferably from 0.1 to 10 μM and, for deep lung deposition, most preferably from 0.1 to 5 μM. If the particles are intended for nasal administration, the preferred mean aerodynamic particle diameter size is approximately 10 μm to 75 μm. The particle size may be measured using an aerosizer (TSI instruments) as will be appreciated by the skilled person.

The microparticles comprise at least two ingredients, a viral particle and a stabilising carbohydrate. Preferably, the viral particle is an envelope virus. The term “envelope virus” refers to any virus which is encapsulated within a membrane, and said membrane contains recognisable, immunogenic molecules. Such molecules include, but are not limited to, proteins, glycoproteins and carbohydrates. Preferably, the envelope viruses are transmitted between hosts through the respiratory route, for example influenza, rubella and mumps viruses. Most preferably the virus is measles.

The virus particle may be in any form, live, live attenuated or killed, provided that the integrity of the antigenic determinants is maintained.

Other suitable live attenuated viruses suitable for use in this invention include West Nile virus, Varicella [chickenpox], rabies, smallpox, MMR, hepatitis A, B, C.

To improve the antigenicity of the viral particles, an adjuvant may be included in the mixture that is spray dried to create the microparticles, so that the resulting microparticle comprises a stabilising carbohydrate, viral particle and adjuvant. Suitable adjuvants include, but are not limited to trehalose acetate, trehalose octapivalate, aluminium salts, squalene mixtures, aquiline mixtures, mercury/peptide, saponin derivatives, muyamyl peptide, mycobacterium cell wall preparations, immunostimulating complexes (ISCOMs) and nonionic block copolymer surfactants, MF59, beta-glucan, heat-labile enterotoxin, cholera toxin, plant lectins and calcium phosphate. For veterinary use, mitogenic components of Freud's adjuvant can be used.

Other additives which may be present in the feedstock include combinations of stabilising polyols, e.g. higher polysaccharides/polymers (for promoting controlled release), magnesium stearate/leucine/trileucine (as lubricants), and phospholipids/surfactants. Blowing agents e.g. volatile salts such as ammonium carbonate, formic acid, etc. may also be included in the feedstock to produce low-density microparticles.

Preferably, the feedstock includes at least one physiologically acceptable salt which reduces water from the resulting composition so that at ambient humidity the vapour pressure of water of crystallisation is at least 2000 Pa at 20° C. and does not interfere with glass formation of the carbohydrate. Such salts are referred to in the art as molecular water-pump buffers (MWPB) and include, for example, ammonium chloride, orthophosphate and sulphate; barium chloride dihydrate, calcium lactate pentahydrate and copper sulphate.

As used herein, the term “stabilising carbohydrate” refers to a carbohydrate that confers stability to the virus. This stability refers to the activity of the virus, and the maintenance of viral particles that are immunogenic. Any physiological acceptable carbohydrate may be used.

Suitable carbohydrates include, but are not limited to, monosaccharides, disaccharides, trisaccharides, oligosaccharides and their corresponding sugar alcohols, polysaccharides and chemically modified carbohydrates such as hydroxyethyl starch sugar copolymers (Ficoll), pullulan, and hydrophobically derivatised carbohydrates (HDC's). Both natural and synthetic carbohydrates are suitable for use herein. Synthetic carbohydrates include, but are not limited to, those which have the glycosidic bond replaced by a thiol or carbon bond. Both D and L forms of the carbohydrates may be used. The carbohydrate may be non-reducing or reducing.

Reducing carbohydrates suitable for use in the present invention are those known in the art and include, but are not limited to, glucose, maltose, lactose, fructose, galactose, mannose, maltulose, iso-maltulose and lactulose.

Non-reducing carbohydrates include, but are not limited to, trehalose, raffinose, stachyose, sucrose and dextran. Other useful carbohydrates include non-reducing glycosides of polyhydroxy compounds selected from sugar alcohols and other straight chain polyalcohols. The sugar alcohol glycosides are preferably monoglycosides, in particular the compounds obtained by reduction of disaccharides such as lactose, maltose, lactulose and maltulose. The glycosidic group is preferably a glucoside or a galactoside and the sugar alcohol is preferably sorbitol (glucitol). particularly preferred carbohydrates are maltitol (4-O-β-D-galactopyranosyl-D-glucitol), lactitol (4-O-β-D-galactopyranosyl-D-glucitol) palatinit (a mixture of GPS, α-D-glucopyranosyl-1-6-sorbitol and GPM, α-D-glucopyranosyl-1-6-mannitol), and its individual sugar alcohols, components GPS and GPM.

Preferably, the carbohydrate exists as a hydrate, including trehalose, lactitol and palatinit. Most preferably, the carbohydrate is trehalose.

It will be appreciated by the skilled person that the microparticles are to be formulated to contain physiologically effective amounts of a virus. That is, when delivered in a unit dosage form, there should be a sufficient amount of the virus to achieve the desired response. As the microparticles of the invention are preferably intended for delivery as dry powders in an inhalation device, it will be appreciated that a unit dose comprises a pre-defined amount of microparticles delivered to the patient in one inspiratory effort. In a preferred embodiment, the microparticles are prepared as single unit dosage forms for inclusion in dry powder inhalers.

The amount of virus present in each microparticle will be determined on the basis of the immunogenicity exhibited by the virus. The amounts can be controlled simply by regulating the concentration of the virus in solution with the primer prior to the spraying step.

The microparticles are intended primarily for delivery by inhalation. The preferred delivery system is a dry powder inhaler (DPI), which relies entirely on the patient's inspiratory efforts to introduce the microparticles in a dry powder form into the lungs. However, alternative inhalation devices will also be used. For example, the microparticles may be formulated for delivery using a metered dose inhaler (MDI), which usually requires the high vapour pressure propellant to force the microparticles into the respiratory tract.

The microparticles may also be administered to the pulmonary tract by nebulisation. Alternatively, the microparticles may be delivered by any suitable route including (but not limited to) nasal, ocular and ballistic (delivery through the skin) methods. For ballistic delivery, the preferred particle size range is 0.1-250 μm.

It is envisaged that the microparticle dry powders may be produced as an intermediate for further processing, due to their surprising stability, and may be reconstituted to a form suitable for injection (via any suitable route, including subcutaneous, intramuscular and intraperitoneal)

The microparticle dry powder may be suspended in a non-aqueous medium, preferably perfluorocarbons or oils to give a stable particle in liquid (PIL) formation. The non-aqueous medium must be a bio-compatible continuous phase liquid. Since carbohydrate stabilisation of the virus is utilised, it is clear that the non-aqueous liquid must be a non-solvent for the carbohydrate. For example any non-aqueous non-toxic oil approved for parenteral use could be used. A low viscosity oil such as ethyloleate is suitable and has the advantage that it is easy to inject. Other suitable oils include (but are not limited to) sesame oil, arachis oil, soya oil and mineral oil.

Water miscible non-aqueous solvents such as glycerol, ethylene glycol, propylene glycol, propylene oxide and polypropylene glycol may also be used as a non-aqueous medium in which to suspend the microparticles.

Perfluorocarbons are extremely stable liquids that are neither hydrophilic nor lipophilic. These may be used as the non-aqueous medium for microparticle suspension as described in International Patent Publication WO 02/32402. Suitable perfluorocarbons will be apparent to one skilled in the field, and may include perfluorooctyl bromide, perfluorophenoethrene, perfluorohetane and perfluorodecalin. This method of suspension is well known in the art, as disclosed by International Patent Publications WO 98/41188, WO 02/32402 and WO 02/066005. Such preparations are suitable for nebulisation, injection (via any suitable route, including subcutaneous, intramuscular and intra peritoneal) or ballistically into, through or across the skin using a liquid jet injector, such as the Mediject and Bioject devices. These preparations do not require refrigeration since they are highly stable and may be formulated as a unit-dose vaccine that does not require reconstitution at the point of administration. The advantages of such preparations are clear.

The microparticles may also be formulated as a powder and admixed with other excipients prior to forming into tablets/capsules/sachets, etc. The sachets may be used to prepare a drink for those with difficulty in swallowing. Such oral dosage forms are particularly suitable for vaccine regimens for polio, rotavirus, etc. Furthermore, the dried live attenuated virus material may be dispersed in a molten wax base and formed into suppositories, pessaries, cervical rings, etc. Such preparations may be useful for inducing mucosal immunity against HIV, Herpes Simplex Virus and especially Human Papillomavirus (HPV 16 and 18) for the prevention of cervical cancer. Similarly, a particle-in-liquid format may be adapted into a spray, designed to be applied to the mouth, vagina or anus, with the aim of stimulating mucosal immunity for protection against sexual transmission. The powders could also be used, in dry form or as a particle-in-liquid dosage form, for intranasal delivery against respiratory syncytial virus (RSV) subgroups A and B, human metapneumovirus (HMPV), and three parainfluenza viruses (PIV1, -2, and -3), which account for up to 55-60% of serious respiratory tract infection in children and infants less than one year of age.

The microparticles will preferably be prepared (spray dried) and packaged using aseptic conditions.

The invention also encompasses the virus containing microparticle dry powder produced by the method described, its use in a method of therapy, and its use in the manufacture of a vaccine for the prevention of a viral infection. Preferably, this infection is measles.

The following examples illustrate the invention.

### Example 1

Schwarz strain measles virus was thawed at 37° C. and added to a filter sterilised formulation (Acrodisc, 0.45 mM) comprising of L-histidine (50 mM, Fischer), L-arginine (50 mM, Sigma), L-alanine (50 mM, Acros) in sterile water. Samples of the measles stock were taken and stored at −70° C. for calculation of loss upon manufacture. The spray dryer was heated to the required inlet temperature, then left for a further 30 min to equilibrate. Liquid formulations were kept on ice during the process.

The concentration of trehalose was varied. In order to optimise the concentration required. Spray drying was initially performed at an outlet temperature of 130° C. and an atomisation pressure of 3 bar to be sure of producing a dry powder. However, at this temperature and pressure, losses of virus were high. The outlet temperature (the temperature the dried virus is exposed to) and atomisation pressure (producing a sheer force on the virus) was thus lowered in an attempt to raise viral recoveries. Powder characteristics (residual moisture, glass transition and particle size) were examined to monitor product quality.

The glass transition was measured using a Differential Scanning Calorimiter (Perkin Elmer Pyris 1, Perkin Elmer Corporation, USA). Samples of known mass of powder were loaded and sealed into aluminium sample pans at<15% RH. Thermal behaviour was analysed under a nitrogen purge at 10° C./min between 20-130° C.

Residual moisture was assessed using a Karl Fischer coulometer (684 KF). 50-100 mg of powder was sampled at<15% RH into equilibrated vials and capped with previously dried butyl stoppers. Powders were dissolved in 1 ml of formamide, being rotated for 1 hour. 100-200μl of solution was injected into the Karl Fischer reagent cell, and the μg of water measured to ultimately calculate the percentage water content of the powders.

Particle size was analysed using a laser light diffraction particle analyser (Coulter LS 230, micro volume module). 20 mg of powder was ground using a mortar and pestle in 1 ml Medium Chain Tri-glyceride (MCT) oil. This was then transferred to a Coulter microvolume cell to achieve an obscuration of between 8-11% (approximately 100 μl). Particle size was expressed as volume median diameter (VMD).

SEM analysis was performed using the gold sputter technique (2-3 nm thickness) and imaged using a Hitachi S3000H PC SEM (Hitachi Scientific Instruments Ltd, CA, US). Analysis was performed at a working distance of 10-11 mm at 15.0 kV.

Table 1 shows the resulting powder characteristics as the outlet is decreased from 87° C. to 28° C.

TABLE 1

| | | | Residual | Glass |
|---|---|---|---|---|
| | Trehalose feed | Particle | moisture | transition |
| Inlet/ | concentration | size | content | temperature |
| Outlet (° C.) | (w/v %) | (MVD, μm) | (% w/w) | (° C.) |
| 130/87 | 2 | 3.2 | 1 | 97 |
| 110/74 | 2 | 3.6 | 1.5 | 93 |
| 60/34 | 2 | 2.6 | 3 | 75 |
| 50/28 | 2 | 4.7 | 2.6 | 74 |
| 30/28 | 10 | 4.7 | 2.6 | 77 |
| 30/28 | 50 | 5.6 | 2.4 | 76 |

To reduce the extent to which powder quality was compromised by this lowering of the outlet temperature and atomisation pressure, optimisation of the spray drying parameters was needed (FIG. 1). Optimisation involved 1) Dramatically reducing the feed rate into the spray drier by exchanging pumps. This provided more energy/volume of feed within the system, allowing a drier end product. 2) Increasing drying air pressure (and thus flow) to give more, energy/volume of feed.

The process conditions are shown in Table 2.

TABLE 2

| | |
|---|---|
| Drying air | Atomisation air |

| Inlet | Outlet | Pressure | Flow rate | Pressure | Flow Rate | Feed rate |
|---|---|---|---|---|---|---|
| (° C.) | (° C.) | (bar) | (L/sec) | (bar) | (L/sec) | (g/min) |
| 130 | 88 | 1.5 | 4.8 | 3 | 0.5 | 2^{a} |
| 110 | 70 | 1.8 | 5 | 3 | 0.5 | 2.5^{a} |
| 60 | 34 | 1.5 | 5 | 3 | 0.5 | 2.7^{a} |
| 50 | 28 | 1.5 | 5 | 3 | 0.5 | 2.5^{a} |
| 30 | 28 | 2 | 6 | 1.5 | 0.23 | 0.25^{b} |

After drying, powders were harvested and stored in<10% RH.

These modified parameters allowed both the spray drying without the need of a heater box and a 50% trehalose solution to successfully occur. These both gave particles suitable for pulmonary delivery.

It can be seen from FIG. 1, using a low trehalose feed concentration with both high and low outlet temperatures, virus losses are considerable. Only once the trehalose concentration is increased and atomisation pressure is reduced, can there be seen a rise in virus potency recovered after initial manufacture of the powder. Virus recoveries are increased further using higher trehalose concentrations in the feed at the same pressure. This confirms that an increase in trehalose concentration is responsible for a rise in virus recoveries, possibly being aided in part by a reduction in atomisation pressure.

### EXAMPLE 2

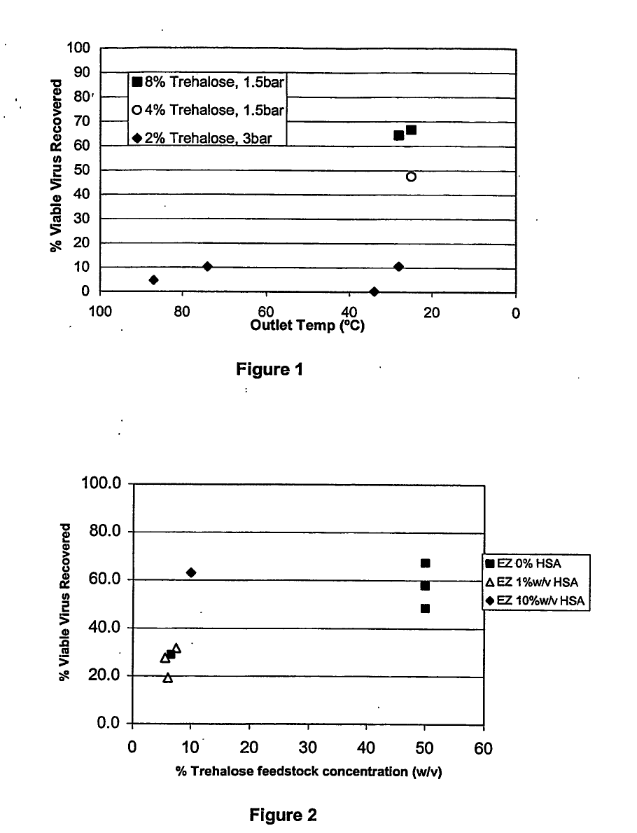

Table 3 and FIG. 2 show the post-process recoveries of the EZ strain using varying concentrations of trehalose and HSA dried under identical optimised conditions as Schwarz strain, above, with an outlet temperature of 28° C.

TABLE 3

| Formulation (w/v) | log₁₀ loss |
|---|---|
| 10% Trehalose | 1.5 (1.4) |
| 10% Trehalose | 1.8 |
| 10% HSA | |
| 50% Trehalose | 0.7 |
| 50% Trehalose | 1.5 |
| 1% HSA | |

As with the Schwarz strain, increasing the trehalose concentration at low temperatures improves virus recoveries. However, the EZ strain improves at a much slower rate, i.e. a 60-70% recovery was achieved at only an 8% w/v trehalose concentration with Schwarz, compared to the same recovery with EZ at a concentration of 50% w/v. No significant difference in virus viability was observed when 1% w/v HSA was added. However, when the concentration was increased to 10% w/v, recoveries were approximately doubled.

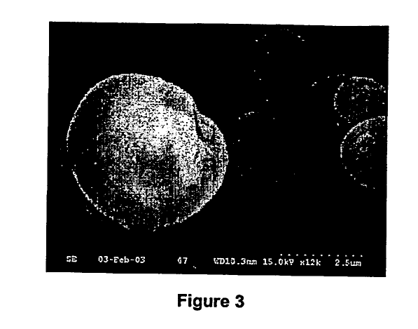

At low concentrations of trehalose in the feedstock (below 10% w/v), stability was poor in both strains, as shown by a 2 log₁₀ loss of over 7 days at 37° C. Upon increasing the trehalose concentration to 50% w/v, stability of the EZ strain virus increased (see Table 3). Stability was greatest over 7 days at 37° C. by the formulation containing no HSA and being spray dried from a 50% trehalose solution. This batch was tested 42 days (6 weeks) after being stored at 4° C. and exhibited only a 0.3 log₁₀ loss. Powder characteristics for this batch are seen in Table 4, and scanning electron microscopy (SEM) of the particles in FIG. 3.

TABLE 4

| | 0% HSA | 1% w/v HSA |
|---|---|---|
| Mean emitted | 72.5 | 85.2 |
| St Dev | 6.53 | 2.83 |
| N= | 6 | 6 |
| % RH when shot | 30-48 | 27-44 |

The 10% w/v trehalose formulation containing no HSA was also stored in Nitrogen as the inert packaging gas, but no significant difference was seen in stability. It is noted that the least stable formulation upon storage is the one with the highest concentration of HSA. This is in contrast to process losses where the increased HSA content raised recoveries significantly.

This data indicates that HSA is a protectant upon process, rather than a storage stabiliser.

### EXAMPLE 3

The emitted dose of two formulations, namely an 8% w/v trehalose formulation with/without 0.5% w/v HSA from a simple dosing device (Penn Century, FIG. 4) were examined. This device is used in animal studies to avoid the facial cavity and trachea, providing direct access to the bifurcation (corina) of the bronchi.

The emitted dose was calculated using the Penn Century (DP-4 dry powder insufflator, 12 cm straight delivery tubes) and recording masses (to 0.01 mg) of the device before and after emission of the powder, using 2×3 ml shots of air from a syringe in ambient humidity. Powders were stored in this case as 5.5 mg±2 mg in HPLC vial inserts packaged under low humidity (<15% RH) and stored at 4° C. until required. Emission from this device was satisfactory in the formulation containing no HSA, although it did have a high standard deviation suggesting inconsistent dosing masses. Emission data from the formulation containing HSA was superior, giving a high and relatively consistent dose each time.

1. A method for producing a micro-particle dry powder comprising a viral particle, comprising the steps of:
spray-drying a mixture of the viral particle and a stabilizing carbohydrate using an outlet temperature of no more than 60° C.

2. The method according to claim 1, wherein the stabilizing carbohydrate is trehalose.

3. The method according to claim 1, wherein the concentration of the carbohydrate is from 2% w/v to 70% w/v.

4. The method according to claim 1, wherein the concentration of the carbohydrate is from 30% w/v to 60% w/v.

5. The method according to claim 1, wherein the concentration of the carbohydrate is from 40% w/v to 55% w/v.

6. The method according to 1, wherein the concentration of the carbohydrate is from 6% w/v to 12% w/v.

7. The method according to claim 1, wherein the spray dryer has an outlet temperature from 20 to 40° C.

8. The method according to claim 1, wherein the feed rate of the spray dryer is from 0.05 to 2 g/min.

9. The method according to claim 1, wherein the spray dryer nozzle-tip configuration is 1 bar 10L/sec to 3 bar 30L/sec.

10. The method according to claim 1, wherein the spray dryer nozzle-tip configuration is 1.5 bar 14L/sec.

11. The method according to claim 1, wherein the spray dryer nozzle-tip configuration is 3 bar 22L/sec.

12. The method according to claim 1, wherein the drying air pressure is from 1.5 bar to 3 bar.

13. The method according to claim 1, wherein the drying air flow rate is from 4.8L/sec to 8L/sec.

14. The method according to claim 1, wherein the atomization air flow rate is from 0.10 to 0.6L/sec.

15. The method according to claim 1, wherein the virus is an envelope virus.

16. The method according to claim 1, wherein the virus is measles.

17. A virus-containing micro-particle dry powder obtainable by a method comprising the steps of:
spray-drying a mixture of the viral particle and a stabilizing carbohydrate using an outlet temperature of no more than 60° C.

18. The virus-containing micro-particle dry powder according to claim 17, wherein each micro-particle is suitable for deep lung deposition.

19. The virus-containing micro-particle dry powder according to claim 17, wherein each micro-particle is suitable for bronchiolar and upper pulmonary tract deposition.

20. The virus-containing micro-particle dry powder according to claim 17, wherein the powder is suspended in a non-aqueous medium.

21. The virus-containing micro-particle dry powder according to claim 20, wherein the non-aqueous medium is a perfluorocarbon.

22. The virus-containing micro-particle dry powder according to claim 20, wherein the non-aqueous medium is an oil, selected from the group consisting of:
sesame oil, arachis oil, soya oil, mineral oil and ethyloleate.

23. The virus-containing micro-particle dry powder according to claim 20, wherein the non-aqueous medium is selected from the group consisting of:
glycerol, ethylene glycol, propylene glycol, propylene oxide and polypropylene glycol.

24. A vaccine comprising a virus-containing micro-particle dry powder wherein said powder is obtainable by a method comprising the steps of:
spray-drying a mixture of the viral particle and a stabilizing carbohydrate using an outlet temperature of no more than 60° C. for use in a method of therapy.

25. A method for the treatment or prevention of a viral infection, wherein said method comprises administering, to a patient in need of such treatment, a virus-containing micro-particle dry powder obtainable by a method comprising the steps of:
spray-drying a mixture of the viral particle and a stabilizing carbohydrate using an outlet temperature of no more than 60° C.

26. The method according to claim 25, wherein the infection is measles.

27. The method according to claim 26, wherein the powder is processed in the form of a tablet or capsule.

28. A sachet comprising a virus-containing micro-particle dry powder obtainable by a method comprising a viral particle, comprising the steps of:
spray-drying a mixture of the viral particle and a stabilizing carbohydrate using an outlet temperature of no more than 60° C.
